(19) United States
(12) Patent Application Publication
(10) Pub. No.: US 2012/0226199 A1
(43) Pub. Date: Sep. 6, 2012

(54) THE DEVICE FOR DETECTING AND FOLLOWING UP A COMPLETE OR PARTIAL ANTERIOR CRUCIATE LIGAMENT INJURY BY MEANS OF COMPARING THE TWO LIMBS

(75) Inventors: Stephane Nouveau, L'Huisserie (FR); Henri Robert, Mayenne (FR)

(73) Assignee: Genourob, Montenay (FR)

(21) Appl. No.: 13/502,790

(22) PCT Filed: Oct. 20, 2010
(86) PCT No.: PCT/FR2010/000696

§ 371 Date: May 15, 2012

(30) Foreign Application Priority Data
Oct. 22, 2009 (FR) 0905071

(51) Int. Cl. A61B 5/11

(52) U.S. Cl. 600/595

(57) ABSTRACT

The aim of the invention is to make improvements to the “GNRB®” device for detecting and following up an ACL injury, one improvement comprising a system for positioning the knee at a flexion of 20° to 90°, characterized by two separate seat portions which are pivotable relative to one another (300 and 400) and by a system for measuring the angle of the pivotal movement, and another improvement comprising a foot-supporting assembly (200), characterized by two portions, one of which is movable relative to the other on a shaft of a rotation sensor (204). The device according to the invention enables the seat of the ACL injury to be identified in the event of a partial tear (PL bundle or AM bundle) and enables the rotational instability of the knee to be measured during the translational movement of the tibia relative to the femur.

The field of the invention is that of the techniques for detecting a lesion of one of the knee ligaments. More specifically, the invention relates to an appliance that can be used to measure the automatic internal rotation of the foot upon the anterior translation of the tibia relative to the femur and that can also be used to measure the translation of the tibia relative to the femur in positions ranging from 20° to 90° of flexion of the knee, so as to reveal a partial lesion of one of the two bundles of the anterior cruciate ligament of the knee, and to reveal the rotational instability of the knee.

In the field of the invention, a number of techniques have been proposed by the prior art.

A first technique is notably known which implements an appliance known as “TELOS” (registered trademark). According to this technique, the patient is placed in a sidelying position on a radiographic table, and a snapshot is taken at the knee level (the latter being subjected to a strain exerted on the posterior face of the calf, in order to thus reproduce the anterior drawer).

The “TELOS” an appliance which can be used for mechanical dynamic radiography. It consists of a metallic central frame, on which the limb is positioned with the knee flexed to 25°, with the thigh immobilized on the anterior face (supra-rotilian rest) and on the anterointerior face of the tibia (patient in sidelying position).

A posterior thrust has to be exerted at the height of the posterior tibial margin under scopic control; two forces of 150 N and 250 N are applied and displayed on the handgrip ergometer.

Four snapshots are then taken: two of the injured limb and two of the other limb. The snapshots are compared (it is the differences which emerge therefrom which are analyzed). On these snapshots, a first line is drawn parallel to the tibial plateaus, followed by two lines perpendicular to the first and passing through the posterior edge of the femoral condyles and of the tibial plateaus. The distance between the two perpendicular lines is measured (in mm), which gives the measurement of the anterior drawer of the tibia relative to the femur.

This technique has proved objective in practice, but does, however, present a number of drawbacks.

First of all, a first drawback lies in the use of X-rays, which prevents iterative measurements when following up the patient, whether pre-operative or post-lesion, or even post-operative, to track the trend of the ligamentoplasty, for example.

Another drawback lies in the fact that the two limbs have to be placed very strictly. This is because the placement of the injured limb and that of the non-injured limb have to be identical on both sides: in particular, the condyles have to be superposed (under radioscopy), as do the tibial plateaus, and the limb has to be in a rotationally neutral position.

Moreover, the “TELOS” is renowned for its low sensitivity, because of the reflex contractures of the ischio-tibial muscles in response to the thrust (because of the uncomfortable and of painful position for the patient), which prevent good measurements of the anterior translation of the tibia from being obtained (high rate of false negatives).

Moreover, this technique does not make it possible to detect a partial lesion of the ACL.

Another technique is also known which implements an appliance known as “KT1000” (US patent filed Sep. 1, 1989 U.S. Pat. No. 4,969,471).

This appliance comprises:
a leg support assembly, which comprises a portion supporting the thigh and a portion that is adjustable in length and intended to extend between the knee and the ankle, the angle between the two portions also being adjustable;
a thrust means for pushing on the calf comprising a portion on which the calf is intended to bear, and linked to a handle intended to be actuated by an operator, this handle incorporating a mechanism for adjusting the force applied to the thrust means;
a device for measuring the anterior translation of the tibia relative to the femur.

Using this appliance entails fastening it onto the front of the leg (the patient being in supine position), by strapping using self-gripping tapes, then pulling on the handle of the appliance in order to exert traction on the tibia, the thigh (the femur) being held by a rotilian rest, the knee being positioned with 20° of flexion and the foot being in neutral position using a chock.

The advantages of this appliance are that it is easy transport and it is very quick to set up.

On the other hand, it does present a number of drawbacks.

A major drawback with this appliance lies in the fact that the results obtained are closely linked to the manipulations of the appliance by the operator. In other words, the same operator will obtain identical results with different measurements on one and the same patient having the same lesion only by performing manipulations of the appliance strictly in the same way.

Obviously, such constancy and such regularity in the manipulations are the reserve of highly experienced operators.

That said, this drawback becomes significant when the measurements made by a number of operators have to be compared. In practice, it is found, on this subject, that there is little correlation in the data collected.

Similarly, because of its lack of measurement accuracy, this appliance does not make possible to detect partial lesions of the ACL.

Moreover, such an appliance is not unaffected by the stray muscular contractions that are likely to occur during the measurements, which obviously impacts on the reliability of the appliance.

There is another technique which is also known, which uses an appliance known by the name “GNRB®” (French patent applications No. FR2906706 and No. FR2906699, PCT extension No. PCT/EP2007/060563).

The object of the “GNRB®” device is a device for detecting and following up a lesion (rupture or tear) of the ACL by anterior translation of the tibia relative to the femur comprising a lower limb support, characterized in that it comprises a thrust means for pushing on the posterosuperior face of the calf and a tibia displacement sensor which is positioned on the anterosuperior face of the tibia, on the TTA.

This device provides solutions to the drawbacks detected in the preceding devices:
the results obtained are independent of the quality and/or the accuracy of the manipulations made by the operators because of a thrust means which comprises at least one cylinder driven by a thrust computer, and because of the lower limb strapping means (recording the fastening pressure),
the accuracy of the results is the order of a tenth of a millimeter,
this device allows for a standardization of the positioning of the limb during different recordings,
this device makes it possible, by interpretation of the comparative measurement curves, to comprehend partial ruptures of the ACL,
this device includes at least one surface electrode facing the ischio-tibial muscles and a reference electrode at the level of the lateral or medial edge of the knee making it possible to detect any stray muscular contraction which prevents the measurement of the anterior displacement of the tibia from being determined with precision.

The publication by Doctor Henri Robert, Orthopaedics & Traumatology: Surgery & Research (2009) 95, 171-176.

One of the drawbacks of this device is that it does not make it possible to detect Partial lesions of the different, bundles of the ACL (anteromedial bundle, AM and posterolateral bundle, PL). The measurement of the translational movement of the tibia relative to the femur in a position, of flexion of the knee at 20° makes it possible to detect a rupture of the anteromedial bundle (by comparing the data collected at 60°) and the measurement of the translational movement of the tibia relative to the femur in a position of flexion of the knee at 60° makes it possible to detect a rupture of the posterolateral bundle (by comparing the data at 20°). For partial Lesions, measurement by the “GNRB®” by positioning the knee with different flexions would make it possible to detect the seat of the partial lesion (PL or AM).

Another drawback of this appliance is that it does not make it possible to measure the rotational instability of the knee. The translational movement of the tibia relative to the femur naturally provokes a slight internal rotation of the knee. Since the “GNRB®” diagnostic principle is to measure this rotation on a strapped limb, the automatic rotation is characterized by an internal rotational movement of the foot. Studying this movement would make it possible to objectify the rotational instability of the knee.

This device has other drawbacks:
it has the drawback of not being able to be used on tall people (>2.05 m)
it also has the drawback of not being suited to small people (children).

Generally, the techniques of the prior art have the following drawbacks:
in no case do the systems make it possible to detect the seat of a partial lesion (AM or PL)
in no case is the rotation parameter of the knee in the translational movement taken into account and measured;
in no case are the devices suited to measuring lesions of one of the bundles of the ACL ligament of the knee in children and in tall people.

The aim of the invention is notably to overcome the drawbacks of the prior art.

Another aim of the invention is to provide such a technique which is a refinement of the GNRB® appliance, which retains all the advantageous principles thereof and adds new measurement parameters thereto.

More specifically, the aim of the invention is to propose a more accurate technique for detecting and following up a lesion of a ligament of the knee which makes it possible to detect a lesion of the anteromedial bundle and/or a lesion of the posterolateral bundle and which takes into consideration the rotational instability of the knee.

Another aim of the invention is to provide such a technique which makes it possible to obtain results with a greater accuracy than that obtained with the techniques of the prior art.

Yet another aim of the invention is to provide a technique which is practical to use.

Yet another aim of the invention is to provide such a technique which is of simple design and easy to implement.

Another aim of the invention is to provide such a technique which allows for measurements on the limbs of small people, in particular children,

Another aim of the invention is to provide such a technique which allows for measurements on the limbs of tall people.

These aims, and others which will become apparent hereinbelow, are achieved by virtue of the invention, the subject of which is a device for detecting and following up a lesion (rupture or tear) of a ligament of the knee by anterior translation of the tibia relative to the femur with a device making it possible to objectify the seat of a lesion of the ligament by virtue of a number of tests in different flexion positions, which is in turn associated with a device for recording the rotation of the foot during the translation.

According to an advantageous solution, the device comprises means for flexing (x) the knee of said limb making it possible to have, 0°<x<90°, said device preferentially comprising a portion supporting the seat of the patient and a portion supporting the limb of the patient to which the “GNRB®” is fastened, the two portions being linked together, one by its rear portion and the other by its front portion, the two portions having height adjustment means.

The principle of this device is to measure the translation of the tibia relative to the femur, in positions of flexion of the knee of 0° to 90°.

According to an advantageous solution, said limb support portion includes means for fastening the “GNRB®” (registered trademark) device for measuring the translation of the tibia relative to the femur.

According to an advantageous solution, said support portions can he adjusted in height by a system of powered cylinders, travel distance indicators, driven by a computer.

According to an advantageous solution, said limb support portion has, at its front end and at its rear end, a mutually independent adjustment system which allows for an inclination of this portion of the device which induces a flexion of the knee of the patient.

According to another advantageous solution, the flexion angle of the knee of the patient is measured by a goniometer. When the flexion angle sought by the practitioner (20° to 90°) is obtained, the position is validated and the inclination of the limb support portion relative to the seat portion is recorded: the cylinders transmit their travel position electronically to the microcomputer which records it.

Advantageously, the measurements of the inclination of the limb support portion and the measurements of the flexion angle of the knee of the patient are transmitted in the form of electronic data to the microcomputer which records them.

Advantageously, for each test performed on one and the same patient, the latter is installed in the same conditions as previously, which allows for better follow-up. The flexing parameters are independent of any human intervention, which helps to increase the reliability of the device.

In these different conditions of flexion of the knee, the practitioner induces a test of translation of the tibia relative to the femur with the device for measuring the translation of the tibia relative to the femur (“GNRB”) fastened onto said limb support portion. According to the advantageous solutions of this device, the data are transmitted to a microcomputer and then. interpreted in the form of curves.

With regard to the measurement of the rotational instability, according to an advantageous feature, the device includes a foot support assembly, which is placed on the sliding part of the appliance for measuring the translation of the tibia relative to the femur.

The principle of this device is to determine the rotational instability of the knee of the patients upon a displacement movement of the tibia relative to the femur with different degrees of flexion of the knee, by measuring the automatic internal rotation of the foot of the limb being tested, during this displacement movement of the tibia relative to the femur.

According to an advantageous solution, said foot support consists of two separate portions, one fixed and the other mobile, the rotational movement between the two being measured by a sensor of accelerometer type.

According to an advantageous solution, said sensor supplies data in electronic form to said computer.

In this wav, it is possible to obtain a measurement of the rotational instability of the knee upon a translational movement of the tibia relative to the femur of the order of a tenth of a millimeter.

Other features and advantages of the invention will become more clearly apparent on reading the following description of a preferred. embodiment of the invention, given as an illustrative and nonlimiting example, and from the appended drawings in which;

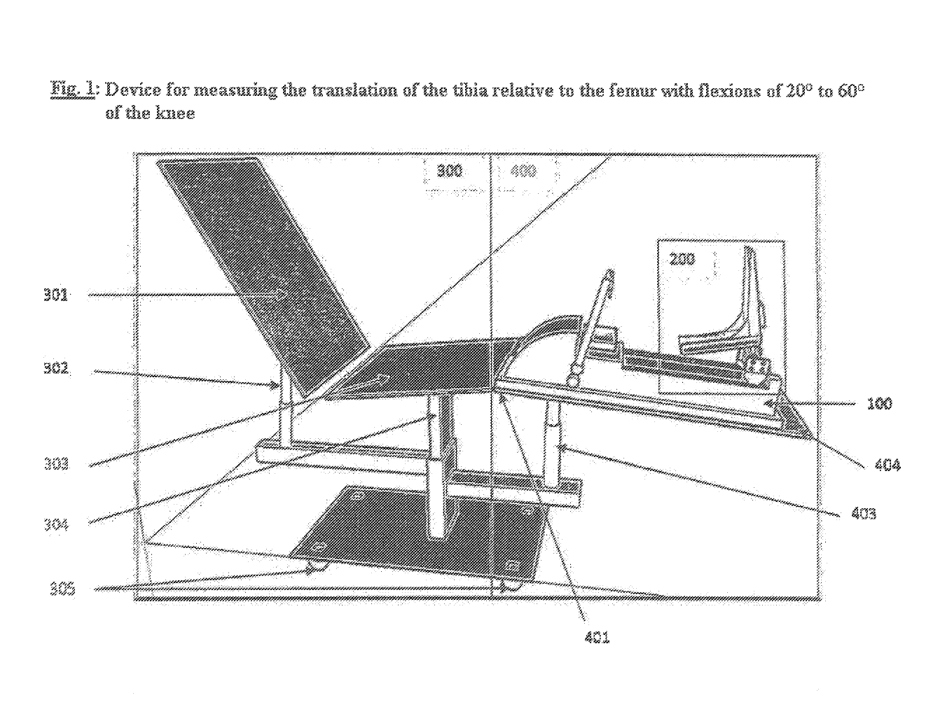

FIG. 1 is an overall view of a device according to the invention;

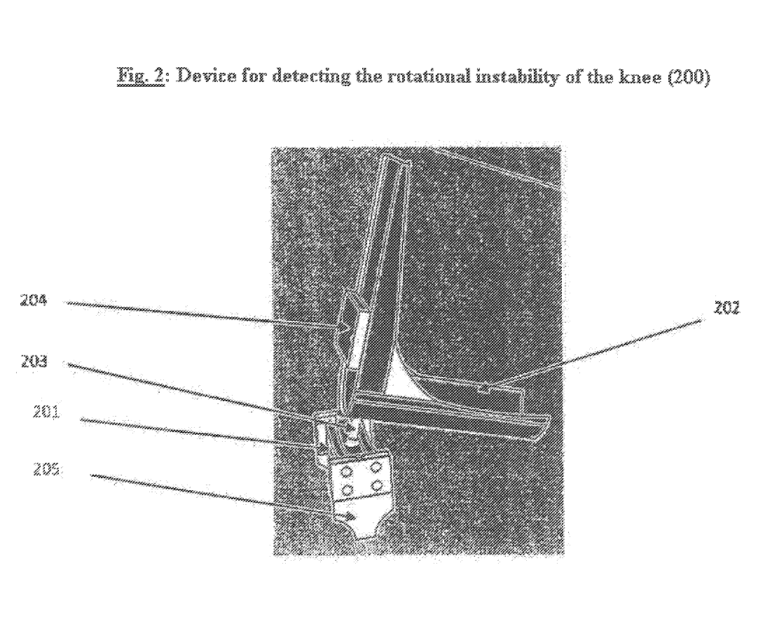

FIG. 2 is a more precise view of the device for recording the rotational instability.

As stated previously, the principle of the invention lies in the fact that it proposes a device for detecting and following up a lesion of the ACL of the knee, being refinements of the existing “GNRB®” device.

With regard to the measurement of the displacement of the tibia relative to the femur in a 0 to 90° positioning, according to an advantageous feature, the device comprises an examination table assembly with removable parts which makes it possible to position the knee of the patient in different flexion positions, in order to determine the seat of a partial lesion of the ACL (PL or AM).

The device comprises a “seat” portion (300) and a “lower limb support” portion (400).

The “seat” portion (300) consists of a seat (303), a back (301) which can be inclined, the inclination can he adjusted by a thrust system consisting of a powered cylinder, travel indicator (302) and an adjustable head rest. This seat portion is made according to the trade practices applied by the manufacturers of medical couches.

This portion (300) is supported by a device which makes it possible to raise and lower the seat, consisting of a number of powered cylinders (304), so as to adapt to the morphology of the patient.

The “lower limb support” portion. (400) consists of a platform (404), manufactured according the trade practices applied by the manufacturers of medical couches, on which is fastened the element for measuring the translation of the tibia and of the femur (100).

This portion (400) is supported by a device which makes it possible to raise, lower and pivot the platform (404), by a number of powered cylinders (403), travel indicators and driven by a microcomputer, in order to obtain the inclination sought by the practitioner.

The portions (300) and (400) are separate: they consist of a device which enables them to be displaced (305) in the form of wheels and a mechanical device (401) which enables the portion (300) to be connected to the portion (400) without limiting the inclination potential of the portion (400).

With regard to the measurement of the rotational instability, according to an advantageous feature, the device comprises a foot support assembly (200), which consists of four separate portions:
A “rotation measuring device support” portion (201) which is fastened to the measuring appliance (100), by a system of mechanical attachments (205) and which comprises a horizontal slideway.
A “foot support” portion (202) on which the foot of the patient is positioned.
A shaft terminated by a ball (203) fastened the foot support portion (202), the ball being able to move in the slideway of the “rotation measuring device support” (201).
A sensor (204) of accelerometer type making it possible to measure the rotation of the shaft (203).

The aim of the invention is to position the foot of the patient on the heel support (202).

This support is attached to the foot and, during the translational movement of the tibia relative to the femur, the slight flexion of the limb that is induced results in an automatic internal rotation of the limb.

This movement results in a rotation of the support shaft (203). The angle of rotation is measured by a sensor (204).

The data are transmitted to a microcomputer which analyzes them so that they can be interpreted.

1-9. (canceled)

10. A device for detecting and following up a lesion of the anterior cruciate ligament (ACL) of the knee including rupture, partial tear, lesion of one of the two LCA bundles, the PL (posterolateral) or the AM (anteromedial) or of the ligamentoplasties of the ACL of the knee, by measuring an anterior translation of the tibia relative to the femur with different knee flexions, and by measuring the automatic rotation of the knee induced by this anterior translational movement of the tibia relative to the femur, wherein it comprises a measuring device, with a support for the lower limb to which the limb of the patient is fastened at knee and ankle level, comprising a thrust means for the posterosuperior face of the calf and a tibia displacement sensor to be positioned on the anterosuperior face of the tibia, on the ATT (anterior tibial tuberosity), fastened onto an examination table incorporating means for positioning the knee with 0° to 90° flexion enabling the measuring device to measure the condition of the lesions of the posterolateral and anteromedial bundles of the ACL, also comprising measuring means, positioned at the level of the lower segment of the limb being tested, for measuring the automatic rotation of the knee induced by the anterior translational movement of the tibia relative to the femur provoked by the measuring device.

11. The device for detecting and following up a lesion of the ACL as claimed in claim 10, wherein it comprises an examination table, which can be adapted to the different patient morphologies, comprising at least two portions that can be inclined relative to one another making it possible to induce a flexion of the knee of 0° to 90° of the patient installed above.

12. The device for detecting and following up a lesion of the ACL as claimed in claim 11, wherein the examination table comprises a first seat portion which consists of a seat comprising a back which can be inclined by a vertical thrust system, and a second lower limb support portion, to which is fastened, by a system of attachments, a device for measuring the anterior translation of the knee relative to the femur, said two portions being placed on a translational thrust system which makes it possible to adapt the height of the armchair to the size of the patients, and the positioning parameters being recorded by a microcomputer making it possible to position the patient identically on each new examination.

13. The device for detecting and following up a lesion of the ACL as claimed in claim 12, wherein the vertical translational thrust system consists of at least two powered cylinders, provided with travel distance indicators, which make it possible to install the patient comfortably in a supine position according to his or her morphology, and to pivot the seat portion relative to the lower limb support portion in order to induce a flexion of the knee by different angulations from 0° to 90°, the positioning parameters being recorded by a microcomputer making it possible to position the patient identically on each new examination.

14. The device for detecting and following up a lesion of the ACL as claimed in claim 11, wherein the two portions of the table are attached, one relative to the other, by a system of mechanical attachments allowing total freedom of one portion relative to the other.

15. The device for detecting and following up a lesion of the ACL as claimed in claim 10, wherein it comprises a device for measuring the automatic rotation of the knee induced by the translational movement of the tibia relative to the femur provoked by a measuring device, comprising two portions that can move relative to one another, to one of which is attached the foot of the limb being tested.

16. The device for detecting and following up a lesion of the ACL as claimed in claim 10, wherein it comprises attachment elements making it possible to insert a device for measuring the automatic rotation of the knee on the sliding portion of the device for measuring the anterior translation of the tibia relative to the femur, inserted on the lower limb support portion of the examination table.

17. The device for detecting and following up a lesion of the ACL as claimed in claim 10, wherein the device for measuring the automatic rotation of the knee comprises a “foot support” portion, to which is fastened, by a system of attachments, the foot and the leg of the limb being tested, which can move on a fixed shaft the “rotation measuring device support” portion, the rotational movement between the two supports being measured by a measuring system.

18. The device for detecting and following up a lesion of the ACL as claimed in claim 10, wherein the system for measuring the rotation of the “foot support” portion relative to the “rotation measuring device support” portion consists of a rotation movement sensor of accelerometer type situated on the shaft which links the two portions of the system for measuring the automatic rotation of the knee, the measured parameters being recorded by a microcomputer.

19. The method for detecting and following up a lesion of the anterior cruciate ligament (ACL) of the knee of a lower limb of a patient, including rupture, partial tear, lesion of one of the two LCA bundles, the PL (posterolateral) or the AM (anteromedial) or of the ligamentoplasties of the ACL of the knee, by measuring an anterior translation of the tibia relative to the femur with different knee flexions, and by measuring the automatic rotation of the knee induced by this anterior translational movement of the tibia relative to the femur, wherein it comprises the steps of:
fastening with a support the lower limb of the patient having a calf, at knee and ankle level,
thrusting the posterosuperior face of the calf,
measuring by a measuring device a tibia displacement with a sensor positioned on the anterosuperior face of the tibia, on the ATT (anterior tibial tuberosity),
positioning the knee of the patient with a flexion at an angle comprised between 0° and 90°, and measuring the condition of the lesions of the posterolateral and anteromedial bundles of the ACL at such angle by measuring via means positioned at the level of the lower segment of the limb being tested, the automatic rotation of the knee induced by the anterior translational movement of the tibia relative to the femur provoked by the thrusting of the calf.

20. The method of claim 19, wherein it comprises the step of inserting a device for measuring the automatic rotation of the knee on a sliding portion of the measuring device, for measuring the displacement of the tibia relative to the femur, inserted on a lower limb support portion of an examination table.

21. The method of claim 19, wherein it comprises measuring the rotation of a “foot support” portion via a rotation movement sensor of accelerometer type situated on a shaft which links two portions of a system used for measuring said automatic rotation of the knee, and recording the measured parameters by a microcomputer.
